(19) United States
(12) Patent Application Publication
(10) Pub. No.: US 2025/0236567 A1
(43) Pub. Date: Jul. 24, 2025

(54) GLOMUS VERSIFORME (GV) MICROBIAL FERTILIZER, AND PREPARATION METHOD AND USE THEREOF

(71) Applicant: Ludong University, Yantai City (CN)

(72) Inventors: Xiaohong GUO, Yantai City (CN); Xiaoya WANG, Yantai City (CN); Bingcheng SI, Yantai City (CN); Wenhao CHEN, Yantai City (CN); Guohui LIANG, Yantai City (CN); Yating LI, Yantai City (CN); Mengxuan WANG, Yantai City (CN); Kai LIU, Yantai City (CN)

(73) Assignee: Ludong University, Yantai City, SD (CN)

(21) Appl. No.: 18/754,307

(22) Filed: Jun. 26, 2024

(30) Foreign Application Priority Data
Jan. 18, 2024 (CN) 202410074105.5

(51) Int. Cl. C05F 11/08; A01G 22/05; C05F 11/02

(52) U.S. Cl. CPC C05F 11/08; A01G 22/05; C05F 11/02

(57) ABSTRACT

A Glomus versiforme (GV) microbial fertilizer, and a preparation method and use thereof are provided. The GV microbial fertilizer includes the following raw materials in parts by weight: 0.5 parts to 1.5 parts of GV, 4 parts to 6 parts of plant ash, and 2 parts to 4 parts of sawdust. Application of the GV microbial fertilizer in the seedling stage of a Solanaceae crop can promote growth and development of the Solanaceae crop and improve yield and quality of the crop. The GV microbial fertilizer can improve nutrients and enzyme activity of soil around a root zone of the Solanaceae crop. The GV microbial fertilizer can also reduce dependence of the Solanaceae crop on chemical fertilizers while maintaining fertility and promoting crop growth by biological processes, which is of great significance to the development of modern and efficient agriculture.

### CROSS-REFERENCE TO RELATED APPLICATION

This patent application claims the benefit and priority of Chinese Patent Application No. 2024100741055 filed with the China National Intellectual Property Administration on Jan. 18, 2024, the disclosure of which is incorporated by reference herein in its entirety as part of the present application.

### TECHNICAL FIELD

The present disclosure belongs to the technical field of microbial fertilizers and particularly relates to a Glomus versiforme (GV) microbial fertilizer, and a preparation method and use thereof.

### BACKGROUND

Fruits and vegetables in the Solanaceae family are called solanaceous crops, mainly including tomatoes, eggplants, and chilies, which have become commonly cultivated summer and autumn vegetables due to strong adaptability, high yield, and rich nutrition. In recent years, solanaceous vegetables have basically achieved annual supply with the rapid development of protected area cultivation. Solanaceous vegetables are high-yield-value crops, and improper fertilization can cause yield decline and economic losses.

After the advent of chemical fertilizers, farmers have developed the habit of applying chemical fertilizers when planting solanaceous crops due to high nutrient content and ease of application. Chemical fertilizers have caused soil hardening and secondary salinization in some areas, leading to insufficient organic matter content, reduced water and fertilizer retention and air permeability, the expansion of medium- and low-yield fields, eutrophication of rivers and lakes, and excessive nitrate content in agricultural products. The migration of farmland nitrogen into the atmosphere damages the ozone layer, causing frequent natural disasters. The use of chemical fertilizers and the expansion of cultivated land can indeed increase the production of solanaceous crops, while the adverse effects on soil are irreversible.

Compared with traditional chemical fertilizers, arbuscular mycorrhizal fungi (AMF)-based bacterial fertilizers have the functions of improving soil, fixing nitrogen, decomposing phosphorus and potassium, and improving crop quality. AMF belongs to the Glomeromycotina phylogenetically and can form a mutually beneficial symbiotic relationship, arbuscular mycorrhiza (AM), with the roots of not less than 80% of plant species on land. This co-evolutionary relationship is generally beneficial to both parties. For example, Zhang Xia et al. investigated the application effect of AM fungi on tomatoes and proved that the arbuscular mycorrhizal Rhizophagus irregularis CD-1 can form a desirable symbiotic relationship with different tomato varieties to significantly promote the growth and development of tomato plants, thus exhibiting excellent application values. Therefore, the development of AMF fertilizer products that are more suitable for planting solanaceous fruits and promoting better growth and yield can further progress the production of solanaceous fruit crops and improve economic benefits.

### SUMMARY

In view of this, an objective of the present disclosure is to provide a GV microbial fertilizer, and a preparation method and use thereof. The GV microbial fertilizer can promote the growth of Solanaceae crops, increase yield and quality of fruits, and improve nutrients and soil enzyme activity of soil.

To achieve the above objective, the present disclosure provides the following technical solutions.

The present disclosure provides a GV microbial fertilizer, including the following raw materials in parts by weight: 0.5 parts to 1.5 parts of GV, 4 parts to 6 parts of plant ash, and 2 parts to 4 parts of sawdust.

The present disclosure further provides a preparation method of the GV microbial fertilizer, including the following steps: inoculating the GV into a leguminous green manure crop serving as a carrier to allow symbiotic culture for 1 to 2 months, removing an overground part of the leguminous green manure crop, and pulverizing a remaining underground part of the leguminous green manure crop to allow mixing with the plant ash and the sawdust in parts by weight.

Preferably, a substrate of the symbiotic culture is turfy soil.

Preferably, the symbiotic culture is conducted at 25° C.±2° C. with a humidity of 60%±5%.

The present disclosure further provides the use of the GV microbial fertilizer in cultivation of a Solanaceae crop.

Preferably, the Solanaceae crop includes a tomato, an eggplant, and a chili.

Preferably, the GV microbial fertilizer is applied at (50-70) g/plant.

Preferably, an application method of the GV microbial fertilizer includes: digging a dressing furrow with a depth of 8 cm to 12 cm at 10 cm to 20 cm around a main root during a seedling stage of the Solanaceae crop, and spreading the GV microbial fertilizer evenly into the dressing furrow.

Preferably, the GV microbial fertilizer promotes the growth of the Solanaceae crop, increases yield and quality of fruits, and improves nutrients and enzyme activity of the soil.

Compared with the prior art, the present disclosure has the following beneficial effects:

The present disclosure provides a GV microbial fertilizer with a simple preparation method. The application of the GV microbial fertilizer to Solanaceae crops can promote the growth and development of the Solanaceae crops and improve growth indicators of the overground part and root system. The GV microbial fertilizer can improve the yield and quality of Solanaceae crops. The GV microbial fertilizer can also improve the soil environment of Solanaceae crops and increase nutrients and enzyme activity of soil around the root zone. The application of GV microbial fertilizer can also reduce the dependence of the Solanaceae crops on chemical fertilizers while maintaining fertility and promoting plant growth by biological processes, which is of great significance to the development of modern and efficient agriculture.

### BRIEF DESCRIPTION OF THE DRAWINGS

FIG. 1 shows the influences of different AMF on tomato plant height;

FIG. 2 shows the influences of different AMF on tomato stem diameter;

FIG. 3 shows the influences of different AMF on tomato yield characteristics;

FIG. 4 shows the changing characteristics of soil urease activity in the tomato root zone under different AMF;

FIG. 5 shows the changing characteristics of soil sucrase activity in the tomato root zone under different AMF;

FIG. 6 shows the changing characteristics of soil catalase activity in the tomato root zone under different AMF; and

FIG. 7 shows the changing characteristics of soil alkaline phosphatase activity in the tomato root zone under different AMF.

### DETAILED DESCRIPTION OF THE EMBODIMENTS

The present disclosure provides a GV microbial fertilizer, including the following raw materials in parts by weight: 0.5 parts to 1.5 parts of GV, 4 parts to 6 parts of plant ash, and 2 parts to 4 parts of sawdust, preferably 1 part of the GV, 5 parts of the plant ash, and 3 parts of the sawdust. In the present disclosure, the GV microbial fertilizer is a biological conditioner with the GV as a main active ingredient and can promote the growth characteristics and root characteristics of Solanaceae crops at different growth stages, increase yield and quality of fruits, and improve the crop root environment (such as nutrients and enzyme activity of soil). As an implementation manner, the GV is a GV strain BGC XJ08F.

The present disclosure further provides a preparation method for the GV microbial fertilizer, including the following steps: inoculating the GV into a leguminous green manure crop serving as a carrier to allow symbiotic culture for 1 to 2 months, removing an overground part of the leguminous green manure crop, and pulverizing a remaining underground part of the leguminous green manure crop to allow mixing with the plant ash and the sawdust in parts by weight.

In the present disclosure, the leguminous green manure crop is preferably white clover; preferably, a substrate of the symbiotic culture is turfy soil; and more preferably, the turfy soil has been sterilized. As an implementation method, the turfy soil is sterilized at 121° C. for 30 min. The symbiotic culture is conducted preferably at 25° C.±2° C. with a humidity of 60%±5%, more preferably at 25° C. with a humidity of 60%. Preferably, the underground part is pulverized to a particle size of not more than 40 mesh.

The present disclosure further provides the use of the GV microbial fertilizer in cultivation of a Solanaceae crop, where the Solanaceae crop includes preferably a tomato, an eggplant, and a chili, more preferably the tomato.

In the present disclosure, the GV microbial fertilizer is applied at preferably (50-70) g/plant, more preferably 60 g/plant. Preferably, an application method of the GV microbial fertilizer includes: digging a dressing furrow with a depth of 8 cm to 12 cm, preferably 10 cm at 10 cm to 20 cm, preferably 15 cm around a main root during a seedling stage of the Solanaceae crop, and spreading the GV microbial fertilizer evenly into the dressing furrow.

In the present disclosure, the GV microbial fertilizer preferably promotes the growth of the Solanaceae crop, increases the yield and the quality of fruits, and improves nutrients and the enzyme activity of soil.

The technical solution provided by the present disclosure will be described in detail below with reference to the examples, but they should not be construed as limiting the claimed scope of the present disclosure.

### Example 1

A preparation method of the GV microbial fertilizer included the following steps:

Turfy soil was sterilized at 121° C. for 30 min as a substrate for symbiotic culture. White clover was planted as a carrier and inoculated with GV. The symbiotic culture was conducted at 25° C. with a humidity of 60% for 1.5 months. An overground part of the white clover was removed and a remaining underground part was pulverized to a particle size of not more than 40 mesh, and then mixed with plant ash and sawdust at a ratio of 1:5:3 by weight.

### Example 2

A preparation method of the GV microbial fertilizer included the following steps:

Turfy soil was sterilized at 121° C. for 30 min as a substrate for symbiotic culture. Alfalfa was planted as a carrier and inoculated with GV. The symbiotic culture was conducted at 27° C. with a humidity of 55% for 1 month. An overground part of the alfalfa was removed and a remaining underground part was pulverized to a particle size of not more than 40 mesh, and then mixed with plant ash and sawdust at a ratio of 0.5:4:2 by weight.

### Example 3

A preparation method of the GV microbial fertilizer included the following steps:

Turfy soil was sterilized at 121° C. for 30 min as a substrate for symbiotic culture. Vicia sativa was planted as a carrier and inoculated with GV The symbiotic culture was conducted at 23° C. with a humidity of 65% for 2 months. An overground part of the Vicia sativa was removed and a remaining underground part was pulverized to a particle size of not more than 40 mesh, and then mixed with plant ash and sawdust at a ratio of 1.5:6:4 by weight.

### Example 4

A method for applying the GV microbial fertilizer to tomatoes included the following steps:

Tomato seedlings were planted and recovered, and a dressing furrow with a depth of 10 cm was dug 15 cm around the main root of the tomato. The GV microbial fertilizer prepared in Example 1 was evenly spread into the dressing furrow at 60 g/plant.

### Example 5

A method for applying the GV microbial fertilizer to eggplants included the following steps:

Eggplant seedlings were planted and recovered, and a dressing furrow with a depth of 8 cm was dug 10 cm around the main root of the eggplant. The GV microbial fertilizer prepared in Example 1 was evenly spread into the dressing furrow at 50 g/plant.

### Example 6

A method for applying the GV microbial fertilizer to chilies included the following steps:

Chili seedlings were planted and recovered, and a dressing furrow with a depth of 12 cm was dug 20 cm around the main root of the chili. The GV microbial fertilizer prepared in Example 1 was evenly spread into the dressing furrow at 70 g/plant.

### Example 7

### Influences of Different AMF on the Growth of Solanaceae Crop

1. Test materials: tomato was selected as a Solanaceae crop, specifically ‘Omanda 3’; the AMF was GV and Rhizophagus intraradices (RI), provided by Ludong University.

The GV had a deposit number of XJ08F in the Bank of Glomales in China (BGC), and a resource number of 1511C0001BGCAM0057 in the National Microbial Resources Platform of AMF; the RI had a deposit number of BJ09 in the BGC, and a resource number of 1511C0001BGCAM0042 in the National Microbial Resources Platform of AMF.

A preparation method of the GV microbial fertilizer and the RI microbial fertilizer included the following steps:

Turfy soil was sterilized at 121° C. for 30 min as a substrate for symbiotic culture. White clover was planted as a carrier and inoculated with AMF. The symbiotic culture was conducted for 1.5 months. An overground part of the white clover was removed and a remaining underground part was pulverized to a particle size of not more than 40 mesh, and then mixed with plant ash and sawdust at a ratio of 1:5:3 by weight.
2. Experimental design: the experiment was divided into three treatments, namely no application of microbial fertilizer (CK), application of GV microbial fertilizer, and application of RI microbial fertilizer.

During the seedling stage of tomatoes, after recovering the seedlings, a small trench was dug with a depth of 10 cm at 15 cm around the main root of the tomato, and the microbial fertilizer was applied at 60 g/plant only once during the entire growth period.

The remaining fertilizers were water-soluble fertilizers. The fertilizers were dissolved in a fertilizer applicator and applied along with the irrigation water through an irrigation pipeline. The nitrogen fertilizer was urea, the phosphate fertilizer was potassium dihydrogen phosphate, and the potassium fertilizer was potassium sulfate. The specific dosages are shown in Table 1.

TABLE 1
Application amounts of nitrogen, phosphorus, and potassium fertilizers

| | | | | Phosphorus fertilizer (P₂O₅ ≥ 52%) | | | Potassium fertilizer (K₂O ≥ 52%) | | |
|---|---|---|---|---|---|---|---|---|---|
| | Nitrogen fertilizer (N ≥ 46%) | | | Flowering | | | Flowering | | |
| | Flowering | Fruit | | and | Fruit | | and | Fruit | |
| | and fruit set | enlarging | Maturation | fruit set | enlarging | Maturation | fruit set | enlarging | Maturation |
| Group | period | period | period | period | period | period | period | period | period |
| Hectare dosage | 42 | 168 | 70 | 30 | 84 | 36 | 60 | 100 | 40 |
| (kg/ha) | | | | | | | | | |
| Cell dosage | 62.06 | 248.22 | 103.43 | 44.33 | 124.11 | 53.19 | 88.65 | 147.75 | 59.10 |
| (g/6.8 m²) | | | | | | | | | |

The amount of irrigation water was calculated based on the 24-h evaporation value of the E601 evaporation dish in the greenhouse. Whenever the cumulative evaporation reached about 20 mm, watering was conducted. After calculation, the amount of water injected each time was 0.204 m³.
3. Indices and methods

After topping the tomatoes, 3 plants in each plot were randomly selected for measurement. The height of the plant was measured using a tape measure, namely a vertical distance from the bottom to the highest point, and the unit was accurate to 1 mm; diameter of the stem between the second and third leaves of the tomato (stem diameter) was measured using a vernier caliper, and each plant was measured three times and averaged, with the unit accurate to 0.1 mm.

Sampling was conducted during the flowering and fruit set period, fruit enlarging period, and maturity period (two ears of fruit were collected during the maturation period), and 3 tomato plants with similar growth were randomly selected from each treatment. Around the tomato plants to be sampled, a pit 40 cm long and wide separately and 50 cm deep was dug using a small shovel to obtain a complete root system of the tomato plant. The overground part (including root, stem, leaves, and fruits) was placed into a kraft paper bag, weighed separately, and a sum of the obtained weights was used as a fresh weight of the overground plant, cured in an oven at 105° C. for 15 min, and then dried at 70° C. until dry and moisture-free, and a dry weight of the overground part was measured. Larger soil particles were removed from the tomato roots, then the roots were cleaned with a brush, and finally, the tomato roots were rinsed in clean water. After confirming that there were no impurities and after drying naturally, a fresh weight of the roots was weighed, and the total root length, root surface area, root volume, root tip number, and other parameters of the roots were obtained through an Epson V850 Pro root scanner. Then the dry weight measurement steps of the overground part were repeated, the dry weight of the underground part was measured, and a root-to-shoot ratio of tomato was calculated.
4. Test results
4.1. Influence on tomato plant height

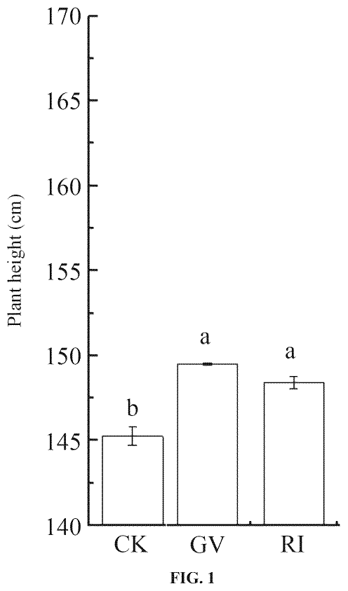

As shown in FIG. 1, there was no significant difference between the treatments (GV and RI) under the inoculation with AMF (P>0.05), but there were obvious differences compared with the CK group, increasing significantly by 2.93% and 2.17% compared with the control group, respectively.
4.2. Influence on tomato stem diameter

As shown in FIG. 2, there was no significant difference between the treatments (GV and RI) after inoculation with AMF (P>0.05).
4.3. Influence on tomato biomass during flowering and fruit set period

TABLE 2
Influences of different treatments on tomato biomass during flowering and fruit set period

| | | Overground | Underground | Underground | |
|---|---|---|---|---|---|
| | Overground part | part dry weight | part fresh | part dry | Root-to-shoot |
| Treatment | fresh weight(g) | (g) | weight (g) | weight (g) | ratio |
| CK | 1473.43 ± 36.89c | 121.85 ± 6.26d | 22.75 ± 4.10f | 6.98 ± 1.38d | 0.18 ± 0.03d |
| GV | 1560.62 ± 53.33bc | 138.14 ± 5.37bc | 34.34 ± 1.69de | 10.84 ± 0.31bc | 0.30 ± 0.06abc |
| RI | 1670.49 ± 31.16ab | 151.74 ± 4.80a | 44.93 ± 5.26abc | 12.14 ± 1.22ab | 0.27 ± 0.05abcd |

As shown in Table 2, the fresh weight of the overground part in different treatments was RI>GV>CK; the fresh weight of the overground part of the RI group was significantly greater than that of the control group (P<0.05), the GV and RI treatments were increased significantly by 13.37% and 13.66% compared with the CK group, respectively; there was no significant difference between the treatments (GV and RI) when inoculated with AMF (P>0.05). The dry weight of the overground part was RI>GV>CK, and the dry weight of the overground part of the treated tomatoes was significantly greater than that of the control group (P<0.05); among the treatments (GV and RI) inoculated with AMF, the RI treatment was significantly increased by 9.85% compared with the GV treatment, which was significantly different from the CK group and significantly increased by 13.37% and 24.53% compared with the control group, respectively. The fresh weight of the underground part was RI>GV>CK, the fresh weight of the underground part of the treated tomatoes was significantly greater than that of the control group (P<0.05); among the treatments (GV and RI) inoculated with AMF, the RI treatment was significantly increased by 30.84% compared with the GV treatment, which was significantly different from the CK group and significantly increased by 50.95% and 97.49% compared with the control group, respectively. The dry weight of the underground part was RI>GV>CK, the dry weights of the underground parts of the GV group and the RI group were significantly greater than that of the control group (P<0.05) and significantly increased by 55.30% and 73.93% compared with the CK group, respectively. The root-to-shoot ratio was GV>RI>CK; the root-to-shoot ratio of the GV group was significantly greater than that of the control group (P<0.05), and was significantly increased by 66.67% compared with the CK group; under the inoculation with AMF, there was no significant difference between the treatments (GV and RI) (P>0.05).
4.4. Influence on tomato biomass during fruit enlarging period

TABLE 3
Influences of different treatments on tomato biomass during fruit enlarging period

| | | | Underground | Underground | |
|---|---|---|---|---|---|
| | Overground part | Overground part | part fresh | part dry | Root-to-shoot |
| Treatment | fresh weight (g) | dry weight (g) | weight (g) | weight (g) | ratio |
| CK | 2680.45 ± 8.24c | 241.06 ± 11.76e | 28.11 ± 1.75d | 8.59 ± 0.78d | 0.10 ± 0.01c |
| GV | 3227.25 ± 145.43b | 286.07 ± 24.75bcd | 51.78 ± 0.83ab | 16.08 ± 1.75ab | 0.18 ± 0.02ab |
| RI | 2820.68 ± 149.83c | 253.14 ± 9.94de | 40.32 ± 1.88c | 12.34 ± 1.94c | 0.14 ± 0.03bc |

As shown in Table 3, the fresh weight of the overground part in different treatments was GV>RI>CK; the fresh weight of the overground part of the GV group was significantly greater than that of the control group (P<0.05), and was increased significantly by 22.88% compared with the CK group; under the inoculation with AMF, in each treatment (GV and RI), the GV group and the RI group were significantly different from the CK group (P>0.05); the dry weight of the overground part was GV>RI>CK; the dry weight of the overground parts in the GV group was significantly greater than that of the control group (P<0.05), which was significantly increased by 18.67% compared with the CK group. In each treatment (GV and RI) under the inoculation with AMF, there was no significant difference between GV treatment and RI treatment (P>0.05). The fresh weight of the underground part was GV>RI>CK; the fresh weight of the underground part of the treated tomatoes was significantly greater than that of the control group (P<0.05); among the treatments (GV and RI) inoculated with AMF, the GV treatment was increased significantly by 28.42% compared with the RI treatment. The dry weight of the underground part was GV>RI>CK, and each group was significantly greater than the control group (P<0.05), increased by 87.19% and 43.66% compared with the CK group, respectively. In the case of AMF vaccination, there was a significant difference between the GV group and the RI group (P<0.05), and the GV treatment was significantly increased by 30.31% compared with the RI treatment. The root-to-shoot ratio was GV>RI>CK, and the GV group was significantly greater than the CK group (P<0.05) and increased by 80.00% compared with the CK group. There was no significant difference between the RI group and the RI group in the case of AMF inoculation (P>0.05).
4.5. Influence on tomato biomass at maturation period

TABLE 4
Influences of different treatments on tomato biomass during maturation period

| | | Overground | Underground | Underground | |
|---|---|---|---|---|---|
| | Overground part | part dry | part fresh | part dry | Root-to-shoot |
| Treatment | fresh weight (g) | weight (g) | weight (g) | weight (g) | ratio |
| CK | 329.94 ± 87.78d | 41.41 ± 5.21d | 19.04 ± 1.42d | 5.55 ± 0.67e | 0.13 ± 0.013ab |
| GV | 628.11 ± 39.23bc | 65.55 ± 4.43bc | 30.63 ± 0.80abc | 8.85 ± 0.86bcd | 0.14 ± 0.010ab |
| RI | 387.16 ± 66.79d | 54.48 ± 9.00cd | 21.33 ± 4.32bcd | 6.63 ± 0.87de | 0.12 ± 0.008b |

As shown in Table 4, the fresh weight of the overground part in different treatments was GV>RI>CK; the GV group was significantly greater than the CK group (P<0.05), increased by 90.37% compared with the CK group; in the case of inoculation with AMF, the GV group was significantly increased by 66.22% compared with the RI group. The dry weight of the overground part was GV>RI>CK; the GV group was significantly greater than the CK group (P<0.05), increased by 58.30% compared with the CK group; there was no significant difference between the treatments (GV and RI) when inoculated with AMF (P>0.05). The fresh weight of the underground part was GV>RI>CK; the GV group was significantly greater than the control group (P<0.05), increased by 60.87% compared with the CK group; there was no significant difference between the treatments (GV and RI) when inoculated with AMF (P>0.05). The dry weight of the underground part was GV>RI>CK; the GV group was significantly greater than the control group (P>0.05), increased by 59.46% compared with the CK group; in the case of inoculation with AMF, there was no significant difference between the treatments (GV and RI) (P>0.05). There was no significant difference in the root-to-shoot ratios between the GV, CK, and RI groups.
4.6. Influence on tomato root system during flowering and fruit set period

TABLE 5
Influences of different treatments on total root length, total root
volume, total root surface area, and average diameter of tomato roots

| | Total root length | Total root volume | Total root surface | Average diameter |
|---|---|---|---|---|
| Treatment | (cm) | (cm³) | area (cm²) | (mm) |
| CK | 1188.34 ± 181.31c | 105.41 ± 13.63c | 575.25 ± 26.13c | 0.98 ± 0.04c |
| GV | 1646.67 ± 74.45b | 117.66 ± 15.13c | 800.83 ± 82.48bc | 1.18 ± 0.03a |
| RI | 1466.62 ± 85.79bc | 106.26 ± 12.16c | 701.73 ± 49.07c | 1.13 ± 0.07abc |

As shown in Table 5, the total root length of different treatments was GV>RI>CK; the total root length of treated tomatoes was significantly greater than that of the control group (P<0.05); in the case of AMF inoculation, there was no significant difference between the treatments (GV, RI) (P>0.05), but there were obvious differences compared with the CK group, which increased significantly by 38.57% and 23.42% compared with the control group, respectively. The total root volume was GV>RI>CK; there was no significant difference between the treatments (GV, RI) when inoculated with AMF (P>0.05). The total root surface area was GV>RI>CK; there was no significant difference between the treatments (GV, RI,) under the inoculation with AMF (P>0.05). The average diameter was GV>RI>CK; the average diameter of the GV group was significantly greater than that of the control group (P<0.05), significantly increased by 20.41% compared with the CK group; there was no significant difference between the treatments (GV, RI) under the inoculation with AMF (P>0.05).

TABLE 6
Influences of different treatments on number of links, nodes, root tips, bifurcations, and crossovers of tomato roots

| | | | | Number of | Number of |
|---|---|---|---|---|---|
| | Number of | Number of | Number of root | bifurcations | crossovers |
| Treatment | links (link) | nodes (node) | tips (root tip) | (bifurcation) | (crossover) |
| CK | 7373.67 ± 1557.27d | 5744.33 ± 1126.61d | 1581.00 ± 224.61e | 3555.67 ± 739.66d | 606.67 ± 167.03d |
| GV | 12230.00 ± 1722.12bc | 9409.00 ± 1093.74bc | 2480.00 ± 152.05cd | 5861.67 ± 884.57bc | 1066.33 ± 188.24cd |
| RI | 10764.00 ± 2313.53cd | 8245.67 ± 1684.57cd | 2127.00 ± 349.78de | 5192.33 ± 1105.39cd | 925.33 ± 245.34cd |

As shown in Table 6, the number of links in different treatments was GV>RI>CK; the number of links in the GV group was significantly greater than that in the control group (P<0.05) and significantly increased by 65.86% compared with the CK group. There was no significant difference between the treatments (GV, RI) when inoculated with AMF (P>0.05). The number of nodes was GV>RI>CK; the number of nodes in the GV group was significantly greater than that in the control group (P<0.05), significantly increased by 63.80% compared with the CK group; there was no significant difference between the treatments (GV, RI) under the inoculation with AMF (P>0.05). The number of root tips was GV>RI>CK; the number of root tips in the GV group was significantly greater than that in the control group (P<0.05), significantly increased by 56.86% compared with the CK group; there was no significant difference between the treatments (GV, RI) under the inoculation with AMF (P>0.05). The number of bifurcations was GV>RI>CK; the number of bifurcations in the GV group was significantly greater than that in the control group (P<0.05), significantly increased by 64.85% compared with the CK group; there was no significant difference between the treatments (GV, RI) under the inoculation with AMF (P>0.05). The number of crossovers was GV>RI>CK, and there was no significant difference between them.
4.7. Influence on tomato root system during fruit enlarging period

TABLE 7
Influences of different treatments on total root length, total root
volume, total root surface area, and average diameter of tomato roots

| | | Total root volume | Total root surface | Average diameter |
|---|---|---|---|---|
| Treatment | Total root length (cm) | (cm³) | area (cm²) | (mm) |
| CK | 1446.16 ± 173.95c | 63.43 ± 3.39e | 633.39 ± 67.50d | 0.91 ± 0.08b |
| GV | 2217.92 ± 157.27a | 102.60 ± 8.42bc | 789.34 ± 55.09abc | 0.93 ± 0.04b |
| RI | 1944.64 ± 179.53b | 104.15 ± 6.53b | 861.82 ± 70.49a | 1.12 ± 0.04a |

As shown in Table 7, the total root length of different treatments was GV>RI>CK; the total root length of tomato in the treatment group was significantly greater than that in the CK group (P<0.05), significantly increased by 58.69% and 53.37% compared with the CK group, respectively; in the case of AMF inoculation, there was a significant difference between the RI group and the GV group (P<0.05), and the GV treatment was increased significantly by 14.05% compared with the RI treatment. The total root volume was RI>GV>CK; the GV group and the RI group were significantly greater than the control group (P<0.05), increased by 61.75% and 64.20% compared with the CK group, respectively; in the case of inoculation with AMF, there was no significant difference between the GV group and the RI group (P>0.05). The total root surface area was RI>GV>CK; the GV group and the RI group were significantly greater than the control group (P<0.05) and increased by 24.62% and 36.06% compared with the CK group, respectively. The average diameter was RI>GV>CK; the RI group was significantly greater than the control group (P<0.05), increased by 23.08% compared with the control group; in the case of AMF inoculation, there was a significant difference between the GV group and the RI group (P>0.05), while the RI treatment was increased significantly by 20.43% compared with GV treatment.

TABLE 8
Influences of different treatments on number of links, nodes, root tips, bifurcations, and crossovers of tomato roots

| | | | Number of | Number of | Number of |
|---|---|---|---|---|---|
| | Number of | Number of | root tips (root | bifurcations | crossovers |
| Treatment | links (link) | nodes (node) | tip) | (bifurcation) | (crossover) |
| CK | 9372.67 ± 1523.49c | 7719.00 ± 980.09d | 2600.00 ± 245.36d | 4413.33 ± 591.28d | 704.67 ± 202.36d |
| GV | 16929.67 ± 426.85a | 13096.33 ± 1376.62ab | 3592.67 ± 176.11b | 7956.33 ± 690.88ab | 1546.33 ± 209.21ab |
| RI | 13968.00 ± 1368.28b | 11187.00 ± 1083.57bc | 3425.00 ± 374.81bc | 6654.00 ± 440.91bc | 1107.00 ± 255.96cd |

As shown in Table 8, the number of links in different treatments was GV>RI>CK; each group was significantly greater than the control group (P<0.05) and increased by 52.26% and 49.03% compared with the CK group, respectively; in the case of AMF inoculation, there was a significant difference between the RI group and the GV group (P<0.05). The number of nodes was GV>RI>CK; in the case of AMF inoculation, the GV group and the RI group were significantly greater than the control group (P<0.05) and increased by 69.66% and 44.93% compared with the CK group, respectively. The number of root tips was GV>RI>CK; the GV group and the RI group were increased by 38.18% and 31.73% compared with the CK group, respectively; there was no significant difference between the treatments (GV, RI) when inoculated with AMF (P>0.05). The number of bifurcations was GV>RI>CK; the GV group and the RI group were significantly greater than the control group (P<0.05) and increased by 80.28% and 50.77% compared with the CK group, respectively. The number of crossovers was GV>RI>CK; the GV group was significantly greater than the control group (P<0.05), increased by 119.44% compared with the CK group; in the case of AMF inoculation, the GV treatment was significantly increased by 39.69% compared with the RI treatment.
4.8. Influence on tomato root system at maturation period

TABLE 9
Influences of different treatments on total root length, total root
volume, total root surface area, and average diameter of tomato roots

| | Total root length | Total root volume | Total root surface area | Average diameter |
|---|---|---|---|---|
| Treatment | (cm) | (cm³) | (cm²) | (mm) |
| CK | 711.91 ± 129.77b | 60.38 ± 9.61c | 422.11 ± 90.13c | 1.00 ± 0.04c |
| GV | 1255.84 ± 58.02a | 86.13 ± 44.05bc | 697.63 ± 90.79ab | 1.28 ± 0.06b |
| RI | 861.47 ± 33.95b | 64.84 ± 5.26c | 577.70 ± 100.08bc | 1.25 ± 0.20b |

As shown in Table 9, the total root length of different treatments was GV>RI>CK; the GV group was significantly greater than the control group (P<0.05), increased by 76.40% compared with the CK group; in the case of AMF inoculation, there was a significant difference between the RI group and the GV group (P<0.05), and the GV group treatment was increased significantly by 45.78% compared with the RI group treatment. The total root volume was GV>RI>CK, and the GV group was significantly greater than the control group (P<0.05). The total root surface area was GV>RI>CK; the significance of the GV group was greater than that of the control group (P<0.05), with an increase of 65.27% compared with the CK group; under the inoculation with AMF, there was no significant difference between the treatments (GV, RI) (P>0.05). The average diameter was GV>RI>CK; there was a significant difference between each treatment group and the control group (P<0.05), increased by 28.00% and 25.00% compared with the CK group, respectively; under the inoculation with AMF, each treatment (GV, RI) had no significant difference (P>0.05).

TABLE 10
Influences of different treatments on number of links, nodes, root tips, bifurcations, and crossovers of tomato roots

| | | | | Number of | Number of |
|---|---|---|---|---|---|
| | Number of | Number of | Number of root | bifurcations | crossovers |
| Treatment | links (link) | nodes (node) | tips (root tip) | (bifurcation) | (crossover) |
| CK | 4938.00 ± 577.68d | 3505.00 ± 503.20e | 1110.33 ± 103.82d | 2092.67 ± 150.42d | 274.33 ± 89.79d |
| GV | 8331.00 ± 578.76a | 6574.00 ± 816.71ab | 1554.33 ± 103.54abc | 4163.67 ± 230.17a | 588.33 ± 60.37a |
| RI | 5211.67 ± 207.81cd | 3958.33 ± 315.01de | 1238.33 ± 210.33cd | 2380.33 ± 82.34cd | 338.67 ± 63.21bcd |

As shown in Table 10, the number of links in different treatments was GV>RI>CK; the significance of the GV group was greater than that of the control group (P<0.05), with an increase of 68.71% compared with the CK group; in the case of AMF inoculation, there was a significant difference between the RI group and the GV group (P<0.05). The number of nodes was GV>RI>CK; the significance of the GV group was greater than that of the control group (P<0.05), increased by 87.56% compared with the CK group; in the case of AMF inoculation, there was a significant difference between the RI group and the GV group (P<0.05). The number of root tips was GV>RI>CK; the significance of the GV group was greater than that of the control group (P<0.05), with an increase of 39.99% compared with the CK group; there was no significant difference in each treatment (GV, RI) when inoculated with AMF (P>0.05). The number of bifurcations was GV>RI>CK; the significance of the GV group was greater than that of the control group (P<0.05), with an increase of 98.96% compared with the CK group; under the inoculation with AMF, the GV treatment was significantly increased by 74.92% compared with the RI treatment. The number of crossovers was GV>RI>CK; the significance of the GV group was greater than that of the control group (P<0.05), with an increase of 114.46% compared with CK; under the inoculation with AMF, the GV treatment was increased significantly by 73.72% compared with the RI treatment.

### Example 8

Influences of different AMF on yield and quality of Solanaceae crops
1. Test materials and 2. The experimental design was the same as those in Example 7.
3. Indices and methods

Ripe tomato fruits were picked to determine the yield, single plant yield, and single fruit quality of calibrated tomato plants in each treatment. In addition, three tomato fruits with similar shapes and maturity were selected from each treatment and homogenized using a juicer to determine the fruit quality. Soluble solids were measured using an Abbe hand-held refractometer, titratable acids were measured using the acid-base titration indicator method, vitamin C was measured using high-performance liquid chromatography (HPLC), soluble proteins were measured using the Coomassie Brilliant Blue method, and soluble sugars were measured using the anthrone method.
4. Test results
4.1. Influence on tomato yield

TABLE 11
Influences of different treatments on tomato yield characteristics

| Treatment | Yield per plant (g) | Mass per fruit (g) | Number of fruits per plant (fruit) |
|---|---|---|---|
| CK | 1166.46 ± 275.41d | 150.29 ± 16.55c | 7.67 ± 1.25c |
| GV | 1843.55 ± 171.86bc | 170.61 ± 25.97bc | 11.00 ± 1.63ab |
| RI | 1696.10 ± 237.72c | 197.72 ± 10.30abc | 8.67 ± 1.70bc |

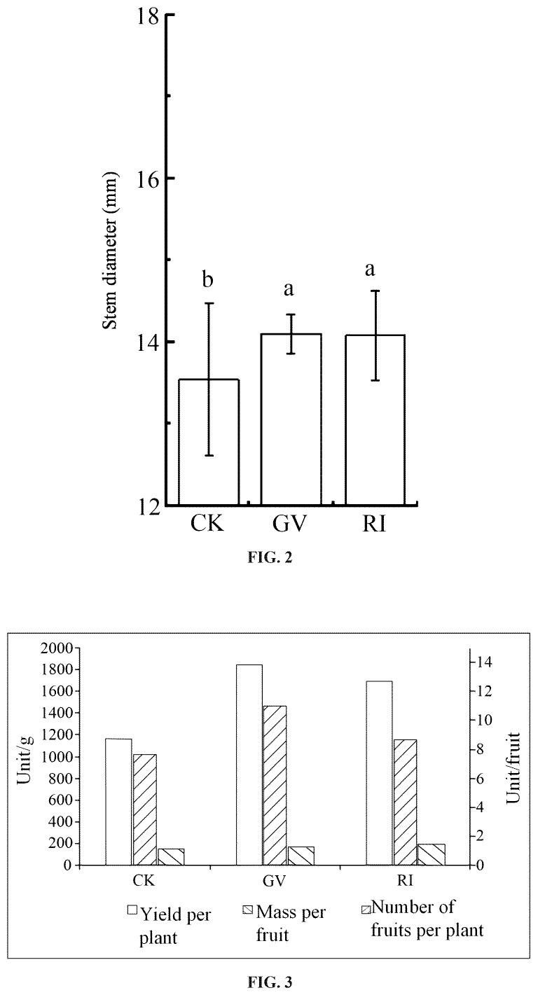

As shown in Table 11 and FIG. 3, the yield per plant in different treatments was GV>RI>CK; the GV group and the RI group were significantly greater than the control group (P<0.05), increased by 58.05% and 45.41% compared with the CK group, respectively; there was no significant difference among the treatments (GV RI) under the inoculation with AMF (P>0.05). The mass per fruit was RI>GV>CK; there was no significant difference among the treatments (GV, RI, GVRI) when inoculated with AMF (P>0.05). The number of fruits per plant was GV>RI>CK; the GV group was significantly greater than the control group (P<0.05), with an increase of 43.42% and 52.15% compared with the CK group; there was no significant difference between the treatments (GV, RI) under the inoculation with AMF (P>0.05).
4.2. Influence on tomato quality

TABLE 12
Influences of different treatments on tomato quality characteristics

| | Soluble | | | | Soluble | |
|---|---|---|---|---|---|---|
| | solids | Titratable | Sugar-to-acid | Vitamin C | proteins | Soluble |
| Treatment | (%) | acid (%) | ratio (%) | (mg/100 g) | (mg/g) | sugars (%) |
| CK | 5.97 ± 0.02cd | 0.26 ± 0.033a | 23.13 ± 3.18cd | 10.65 ± 0.60d | 0.45 ± 0.002d | 1.52 ± 0.007h |
| GV | 6.15 ± 0.12bc | 0.20 ± 0.007bc | 30.84 ± 0.60abc | 12.59 ± 0.72c | 0.51 ± 0.004c | 3.31 ± 0.001b |
| RI | 5.79 ± 0.14d | 0.17 ± 0.006c | 34.89 ± 1.85a | 13.70 ± 0.54c | 0.53 ± 0.001bc | 2.39 ± 0.006g |

As shown in Table 12, the soluble solids in different treatments were GV>CK>RI; under the inoculation with AMF, there were significant differences between the treatments (GV, RI) (P<0.05), and the GV treatment was increased significantly by 6.22% compared with the RI treatment. The titratable acid was CK>GV>RI; under the inoculation with AMF, there was no significant difference between the GV group and RI treatment (P>0.05). The sugar-to-acid ratio was RI>GV>CK; the RI group was increased by 43.93% compared with the CK group; there was no significant difference between the RI group and other treatments when inoculated with AMF (P>0.05). The vitamin C content was RI>GV>CK; the GV group and the RI group were significantly greater than the control group (P<0.05), increased by 18.22% and 28.64% compared with the CK group, respectively; in the case of AMF inoculation, there was no significant difference between the GV group and the RI group (P>0.05). The soluble proteins were RI>GV>CK; the GV group and the RI group were significantly greater than the control group (P<0.05), increased by 13.33% and 17.78% compared with the CK group, respectively; in the case of AMF inoculation, there was no significant difference between the GV group and the RI group (P>0.05). The soluble sugars were GV>RI>CK; each group was significantly greater than the control group (P<0.05), increased by 117.76% and 57.24% compared with the CK group, respectively; under the inoculation with AMF, each treatment (GV, RI,) had significant difference (P<0.05), and the GV treatment was increased significantly by 38.49% compared with the RI treatment.

### Example 9

Influences of different AMF on root soil nutrients and enzyme activity of Solanaceae crops
1. Test materials and 2. The experimental design was the same as those in Example 7.
3. Indices and methods
3.1. Soil nutrients: the soil samples of tomato were collected during the flowering and fruit set period, fruit enlarging period, and maturation period, the stones were removed, the samples were air-dried naturally, passed through a prescribed sieve, placed in a ziplock bag, and the soil nutrients were measured. Soil organic carbon (SOC) was measured using the potassium dichromate oxidation-spectrophotometric method, total nitrogen (TN) was measured using the semi-trace Kelvin method, alkali-hydrolyzable nitrogen (AN) was measured using the alkaline hydrolysis-diffusion method, total phosphorus (TP) was measured by the molybdenum antimony colorimetric method, available phosphorus (AP) was measured by the sodium bicarbonate extraction-molybdenum antimony spectrophotometric method, and available potassium (AK) was measured by the ammonium acetate extraction-flame photometry method.
3.2. Soil enzyme activity: soil samples of tomato were collected during the flowering and fruit set period, fruit enlarging period, and maturation period, impurities were removed, and the samples were air-dried. After passing through a 40-mesh sieve, urease, sucrase, catalase, and alkaline phosphatase in the soil were measured using the Grace biological kit.
4. Test results
4.1. Influence on soil nutrients during flowering and fruit set period

TABLE 13
Soil nutrient characteristics of tomato under different treatments during flowering and fruit set period

| | SOC | | | | | |
|---|---|---|---|---|---|---|
| Treatment | (g/kg) | TP (g/kg) | AP (mg/kg) | TN (g/kg) | AN (mg/kg) | AK (mg/kg) |
| CK | 27.73 ± 1.37c | 2.79 ± 0.024d | 632.63 ± 11.95a | 2.24 ± 0.012c | 156.13 ± 3.21e | 605.67 ± 2.87g |
| GV | 31.67 ± 0.12b | 3.65 ± 0.005a | 536.23 ± 1.18c | 2.71 ± 0.014a | 195.57 ± 2.11b | 774.67 ± 5.79d |
| RI | 25.93 ± 0.48d | 2.87 ± 0.012c | 509.57 ± 4.86d | 2.07 ± 0.009d | 157.93 ± 1.19e | 699.67 ± 3.30e |

As shown in Table 13, under the inoculation with AMF, there were significant differences between the treatments (GV, RI) (P<0.05), and the GV treatment had the highest SOC content, followed by the RI treatment. Under the inoculation with AMF, there were significant differences between the treatments (GV, RI) (P<0.05), and the GV treatment had the highest TP content, followed by the RI treatment. The AP content of soil in each treatment was significantly lower than that of the control treatment (P<0.05). In each treatment (GV, RI) under the inoculation with AMF, the RI group was significantly reduced by 1.05 times compared with the GV group. Under the inoculation with AMF, there were significant differences between the treatments (GV, RI) (P<0.05), and the GV treatment had the highest TN content, followed by the RI treatment. Under the inoculation with AMF, there were significant differences between the treatments (GV, RI) (P<0.05), and the GV treatment had the highest AN content, followed by the RI treatment. The AK content of soil in each treatment was significantly lower than that of the control treatment (P<0.05). Under the inoculation with AMF, there were significant differences between the treatments (GV, RI) (P<0.05), the GV treatment had the highest AK content and the RI treatment had the lowest content.
4.2. Influence on soil nutrients during fruit enlarging period

TABLE 14
Soil nutrient characteristics of tomato under different treatments during the fruit enlarging period

| | SOC | | | | | |
|---|---|---|---|---|---|---|
| Treatment | (g/kg) | TP (g/kg) | AP (mg/kg) | TN (g/kg) | AN (mg/kg) | AK (mg/kg) |
| CK | 24.27 ± 0.17g | 2.49 ± 0.017e | 373.73 ± 2.15g | 2.04 ± 0.009e | 216.87 ± 2.75b | 462.67 ± 2.49f |
| GV | 34.43 ± 0.33b | 2.56 ± 0.017d | 651.37 ± 6.89b | 2.32 ± 0.012c | 105.97 ± 3.39f | 751.67 ± 15.11d |
| RI | 33.23 ± 0.21c | 2.70 ± 0.019c | 687.47 ± 16.64a | 2.38 ± 0.012b | 219.57 ± 1.72b | 791.00 ± 2.83c |

As shown in Table 14, the SOC content of each treatment was significantly higher than that of the control treatment (P<0.05); under the inoculation with AMF, there was a significant difference between the treatments (GV, RI) (P<0.05), where the GV treatment had the highest SOC content, followed by the RI treatment, and the GV group was significantly increased by 1.04 times compared with the RI group. The soil TP content in the GV group was significantly reduced by 1.05 times compared with the RI group. The AP content was highest in the RI treatment, followed by the GV treatment. The soil TN content was significantly different between the treatments (GV, RI) when inoculated with AMF (P<0.05), where the RI treatment had the highest TN content, followed by the GV treatment. The AN content in the soil of the GV group was significantly lower than that of the control group (P<0.05), and was significantly lower than that of the CK group by 1.12 and 2.05 times, respectively; under the inoculation with AMF, there were significant differences between the treatments (GV, RI, GVRI) (P<0.05), where the RI treatment had the highest AN content, and the GV treatment had the lowest content; the GV group was significantly reduced by 2.07 times compared with the RI group. The soil AK content of each treatment was significantly higher than that of the control treatment (P<0.05); in each treatment (GV, RI) under the inoculation with AMF, the RI group was increased significantly by 1.05 times compared with the GV group, had obvious differences compared with the CK group, and was significantly increased by 1.62 times and 1.64 times compared with the control group, respectively.
4.3. Influence on soil nutrients during the maturation period

TABLE 15
Soil nutrient characteristics of tomato under different treatments during maturation period

| Treatment | SOC (g/kg) | TP (g/kg) | AP (mg/kg) | TN (g/kg) | AN (mg/kg) | AK (mg/kg) |
|---|---|---|---|---|---|---|
| CK | 24.97 ± 0.17e | 2.46 ± 0g | 648.43 ± 20.08a | 2.33 ± 0.017d | 225.93 ± 2.50e | 587.33 ± 10.87e |
| GV | 27.43 ± 0.29d | 2.71 ± 0.008d | 511.00 ± 5.61c | 2.11 ± 0f | 210.10 ± 3.50f | 649.33 ± 16.34c |
| RI | 20.23 ± 0.29g | 2.69 ± 0.005e | 437.33 ± 2.46d | 2.15 ± 0.016e | 236.93 ± 3.55d | 628.33 ± 20.04cd |

As shown in Table 15, the SOC content in the RI group was significantly lower than that in the control group (P<0.05), and was significantly lower than that in the CK group by 1.23 times; under the inoculation with AMF, there was a significant difference between the treatments (GV, RI) (P<0.05), where the GV treatment had the highest SOC content, and the RI treatment had the lowest SOC content; the RI group was significantly lower than the GV group by 1.36 times. The soil TP content of each treatment was higher than that of the control treatment; under the inoculation with AMF, there was a significant difference between the treatments (GV, RI) (P<0.05), where the GV treatment had the highest TP content, followed by the RI treatment, and the GV group was significantly increased by 1.01 times compared with the RI group. In the case of AMF inoculation, there were significant differences between the treatments (GV, RI) (P<0.05), where the GV treatment had the highest AP content, followed by the RI treatment; the RI group was significantly reduced by 1.17 times compared with the GV group and had an obvious difference compared with the CK group. The soil TN contents in the GV group and RI group were significantly lower than that in the control group (P<0.05), and were significantly lower than that in the CK group by 1.10 times and 1.08 times, respectively; under the inoculation with AMF, there was a significant difference between the treatments (GV, RI) (P<0.05), where the RI treatment had the highest TN content, followed by GV treatment, and the GV group was significantly reduced by 1.02 times compared with the RI group. The AN content in the soil of the GV group was significantly lower than that of the control group (P<0.05), and was significantly lower than that of the CK group by 1.08 times; under the inoculation with AMF, there was a significant difference between the treatments (GV, RI) (P<0.05), where the RI treatment had the highest AN content, followed by the GV treatment, and the GV group was significantly lower than the RI group by 1.13 times. The soil AK content of each treatment was higher than that of the control treatment; in each treatment (GV, RI) under the inoculation with AMF, there was no significant difference between the GV group and the RI group (P>0.05), and the GV group and RI group were significantly increased by 1.11 times and 1.07 times compared with the control group, respectively.
4.4. Response of urease in soil at different growth stages

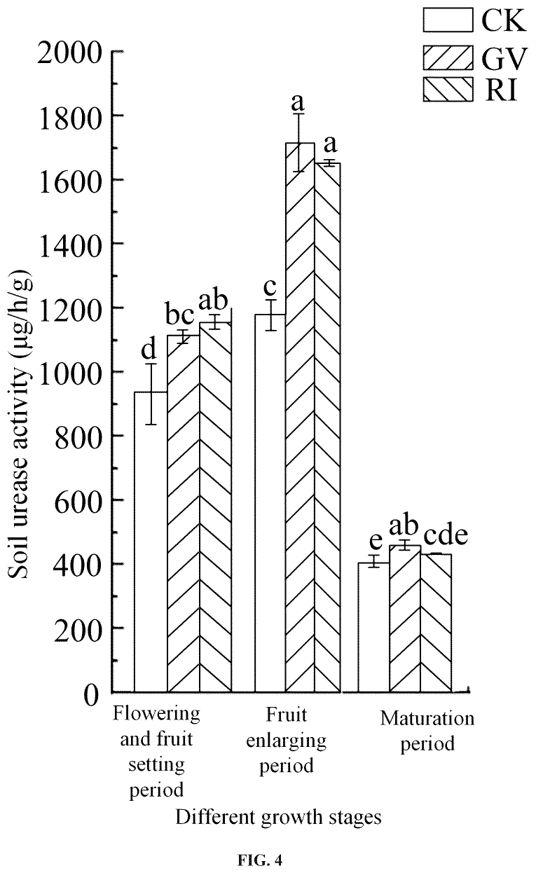

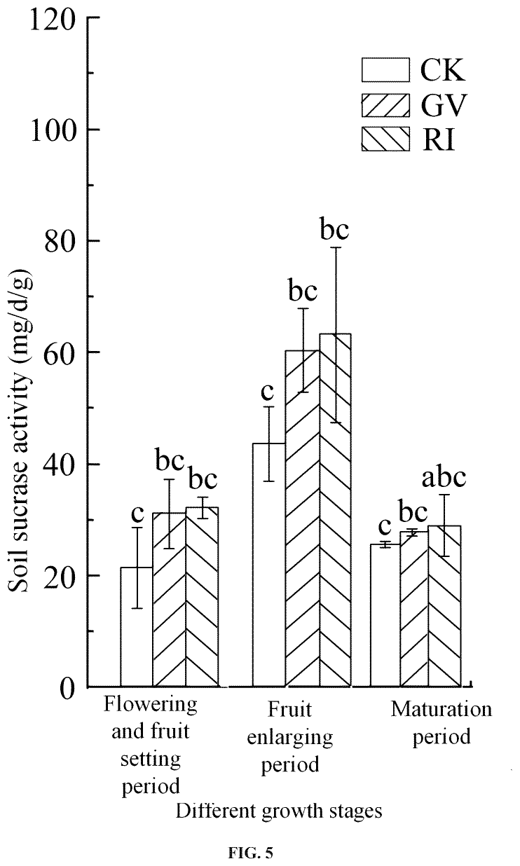

As shown in FIG. 4, during the tomato growth period, the soil urease activity of each treatment showed a trend of increasing and then decreasing with the extension of growth time, and the maximum value appeared during the fruit enlarging period. During the entire growth period, the urease activity of the treated soil was greater than that of the control group. When inoculated with AMF during the flowering and fruit set period, the GV and RI groups significantly increased soil urease activity. When inoculated with AMF during the fruit enlarging period, there was no significant difference between the treatments (GV, RI) (P>0.05), and the two groups were significantly increased by 1.45 times and 1.40 times compared with the control group, respectively.
4.5. Response of sucrase in the soil at different growth stages

As shown in FIG. 5, during the tomato growth period, the soil sucrase activity of each treatment showed a trend of increasing and then decreasing with the extension of growth time, and the maximum value appeared during the fruit enlarging period. During the entire growth period, the sucrase activity of the treated soil was greater than that of the control group. There was no significant difference between the treatments (GV, RI) when inoculated with AMF (P>0.05) during the flowering and fruit set period. When inoculated with AMF during the fruit enlarging period, there was no significant difference between the treatments (GV, RI) (P>0.05), there was no significant difference between the GV and RI groups compared with the control group (P>0.05). There was no significant difference between the treatments (GV, RI) when inoculated with AMF (P>0.05) during the maturation period.
4.6. Response of catalase in the soil at different growth stages

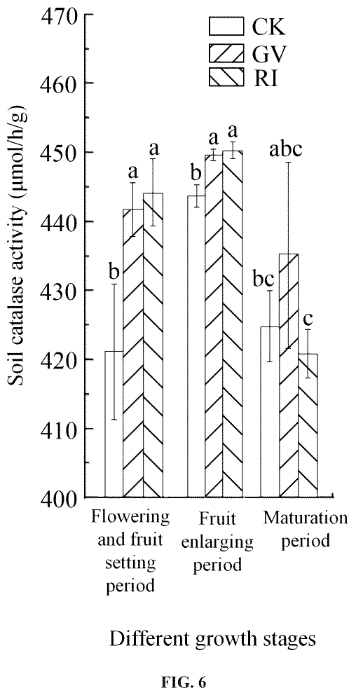

As shown in FIG. 6, during the tomato growth period, the soil catalase activity of each treatment showed a trend of increasing and then decreasing with the extension of growth time, and the maximum value appeared during the fruit enlarging period. During the flowering and fruit set period, the treatment group had no significant difference (P>0.05) but was significantly higher than the control group (P<0.05), and the GV group and RI group were significantly increased by 1.05 times compared with the control group. During the fruit enlarging period, the treatment groups were significantly increased by 1.01 times compared with the control group. During the maturation period, there were no significant differences between the treatment groups and the control.
4.7. Response of alkaline phosphatase in soil at different growth stages

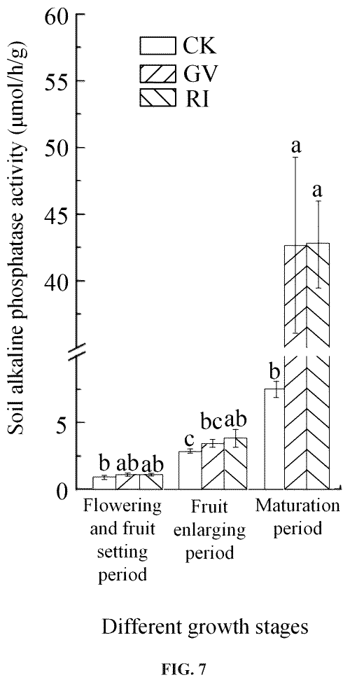

As shown in FIG. 7, during the tomato growth period, the soil alkaline phosphatase activity in each treatment showed an increasing trend with the extension of growth time, and the maximum value appeared in the maturation period. At the flowering and fruit set period, there were no significant differences between the treatment groups and the control (P>0.05). During the fruit enlarging period, the RI group was significantly increased by 1.34 times compared with the control group, and there was no significant difference between other treatment groups and the control (P>0.05). In the maturation period, the treatment groups showed no significant difference and were significantly higher than those in the control group (P<0.05).

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A Glomus versiforme (GV) microbial fertilizer, comprising the following raw materials in parts by weight: 0.5 parts to 1.5 parts of GV, 4 parts to 6 parts of plant ash, and 2 parts to 4 parts of sawdust.

2. A method for preparing the GV microbial fertilizer according to claim 1, comprising the following steps: inoculating the GV into a leguminous green manure crop serving as a carrier to allow symbiotic culture for 1 to 2 months, removing an overground part of the leguminous green manure crop, and pulverizing a remaining underground part of the leguminous green manure crop to allow mixing with the plant ash and the sawdust in parts by weight.

3. The method according to claim 2, wherein a substrate of the symbiotic culture is turfy soil.

4. The method according to claim 2, wherein the symbiotic culture is conducted at 25° C.±2° C. with a humidity of 60%±5%.

5. A method for cultivating a Solanaceae crop, comprising applying the GV microbial fertilizer according to claim 1 in a soil for the Solanaceae crop.

6. The method according to claim 5, wherein the Solanaceae crop comprises a tomato, an eggplant, and a chili.

7. The method according to claim 5, wherein the GV microbial fertilizer is applied at (50-70) g/plant.

8. The method according to claim 5, wherein an application method of the GV microbial fertilizer comprises: digging a dressing furrow with a depth of 8 cm to 12 cm at 10 cm to 20 cm around a main root during a seedling stage of the Solanaceae crop, and spreading the GV microbial fertilizer evenly into the dressing furrow.

9. The method according to claim 5, wherein the GV microbial fertilizer promotes growth of the Solanaceae crop, increases yield and quality of fruits, and improves nutrients and enzyme activity of soil.
